(19) United States
(12) Patent Application Publication
(10) Pub. No.: US 2005/0074869 A1
(43) Pub. Date: Apr. 7, 2005

(54) Hydrodynamic micromanipulation of individual cells to patterned attachment sites

(76) Inventors: Makoto Yoshida, Euclid, OH (US); Koji Tohda, Mayfield Heights, OH (US); Miklos Gratzl, Mayfield Heights, OH (US)

Correspondence Address: Richard J. Minnich;Fay, Sharpe, Fagan, Minnich & McKee, LLP, 7th Floor, 1100 Superior Avenue, Cleveland, OH, 44114-2518, US

(21) Appl. No.: 10/681,545

(22) Filed: Oct. 8, 2003

Related U.S. Application Data
(60) Provisional application No. 60/416,792, filed on Oct. 8, 2002.

(51) Int. Cl. C12M 1/34

(52) U.S. Cl. 435/287.1; 435/287.3; 435/288.7; 435/30

(57) ABSTRACT

A system for manipulation of a cell (100) includes a platform (33) which defines a surface (50) having a site (60) at which the cell has a higher probability of attachment in a period of time than on an adjacent area (52) of the surface. First and second syringes (10, 12) are selectively actuated to deliver a liquid to first and second tubes 26, 28, respectively. Outlets (29, 29′) of the first and second tubes are positioned so as to deliver the liquid to a liquid medium on the platform, thereby creating generally orthogonal fluid flows through the liquid to/from a region of interest (35). Cells located in the liquid medium at the region of interest move at a speed which is much lower than that of the liquid at the tube outlets and along the tube axis, allowing the cell to be manipulated to the site by manual or automated actuation of the syringes. Once on the site, the cell attaches, unwanted cells on the adjacent area can be washed away, allowing electrochemical or other measurements to be performed on an accurately positioned cell or group of cells.

### BACKGROUND OF THE INVENTION

This application claims the benefit of Provisional Application Ser. No. 60/416,792, filed Oct. 8, 2002, which is incorporated herein in its entirety, by reference.

1. Field of the Invention

The invention relates to a method and apparatus for moving selected microscopic particles, such as single cells, small groups of cells, or even beads to a selected location, where the microscopic particle is bound. In particular, it relates to a hydraulic based hydrodynamic manipulation system for controlled navigation of cells onto a hydrophilic site, and will be described with particular reference thereto.

2. Discussion of the Art

To understand the transport of biochemicals, drugs, and other species into and out of biological cells, it is advantageous for research or clinical personnel to be able to isolate a small colony of human or animal cells or even a single cell. For reliable and quantitative data to be attained, it is beneficial for the cell or colony of cells to be located in a defined area of a substrate, such as at a microsensor. Additionally, for some studies, spatial distribution of the cells has an influence on the biological transport characteristics of the cells.

Several different cell manipulation technologies have been developed. Mechanical techniques for cell manipulation include the use of a micropipette or microtweezers. Such techniques tend to damage the delicate cells. Electrical techniques for cell manipulation include those which utilize dielecrophoretic gradient forces. In this technique, inhomogeneous high-frequency electromagnetic fields are created. In these fields cells become polarized with respect to the surrounding aqueous medium and experience net dielectrophoretic effects. The resulting attractive or repulsive forces can be used for cell manipulation. Optical manipulation techniques employ a laser beam having an electromagnetic gradient force which allows the positioning of cells.

Although both electrical and optical trapping can be used to manipulate single cells with good geometrical accuracy, the cost and complexity of the equipment used has prevented their widespread use.

For some studies, such as drug efflux, it would be desirable to attach cells to a substrate as quickly as possible, ideally within a matter of minutes. Many techniques for locating cells rely on biological attachment to the surface of a substrate. This attachment can take several days to achieve and thus for some studies, the delay is unsatisfactory.

The present invention provides a new and improved hydrodynamic cell manipulation system and method of use which overcome the above-referenced problems and others.

### SUMMARY OF THE INVENTION

In accordance with one aspect of the present invention, a method of manipulating a cell is provided. The method includes delivering at least one cell to a liquid medium which extends over a surface. A first fluid flow is applied to the liquid medium from a first direction which moves the cell in a first direction on the surface. A second fluid flow is applied to the liquid medium from a second direction angularly spaced from the first direction which moves the cell in a second direction on the surface.

In accordance with another aspect of the invention, a system for manipulation of a cell is provided. The system includes a platform which defines a surface having a site at which the cell has a higher probability of attachment in a period of time than an adjacent area of the surface. First means selectively deliver a fluid to a first fluid outlet located adjacent the surface, the first outlet being oriented to provide a first fluid flow in a first direction in a liquid medium on the surface. Second means selectively deliver a fluid to second fluid outlet located adjacent the surface, the second outlet being oriented to provide a second fluid flow in a second direction, angularly spaced from the first direction, in the liquid medium on the surface, whereby by actuation of the first and second delivery devices, a cell can be navigated across the adjacent area through the liquid medium to the site.

In accordance with another aspect of the present invention, a system for performing an electrochemical measurement on a cell is provided. The system includes a platform which defines a surface and at least one electrode positioned for performing an electrochemical measurement which is influenced by a cell located on the surface adjacent the electrode. A first tube is positioned to supply a liquid to a liquid medium on the surface toward the electrode in a first fluid flow direction. A second tube is positioned to supply a liquid to the liquid medium on the surface toward the electrode in a second fluid flow direction. Means are provided for selectively controlling fluid flow to the first and second tubes.

In accordance with another aspect of the present invention, a method for forming a cell manipulation system is provided. The system includes connecting a first syringe to a first tube, connecting a second syringe to a second tube; and positioning an outlet of the first tube in a liquid medium adjacent an upper surface of a platform in a first orientation. The platform defines a region of interest including a site for attachment of a cell and an area surrounding the site at which a probability of a cell attaching is less than that at the site. The method further includes positioning an outlet of a second tube in the liquid medium adjacent the upper surface of the platform in a second orientation, such that fluid flows from the first and second tubes intercept at the region of interest. By selectively actuating the first and second syringes, a cell located in the liquid medium in the region of interest can be navigated to the site.

An advantage of at least one embodiment of the present invention is that it enables controlled navigation of single cells or colonies.

Another advantage of at least one embodiment of the present invention is that it enables both pushing and pulling forces to be applied to the cells.

Another advantage of at least one embodiment of the present invention is that unwanted cells are readily removed.

Another advantage of at least one embodiment of the present invention is that it facilitates cellular transport and communication studies.

Another advantage of at least one embodiment of the present invention is that it enables cells to be moved at a velocity of a few micrometers per second.

Still further advantages of the present invention will be readily apparent to those skilled in the art, upon a reading of the following disclosure and a review of the accompanying drawings.

### BRIEF DESCRIPTION OF THE DRAWINGS

FIG. 1 is a schematic side view of a hydrodynamic cell manipulation system according to the present invention;

FIG. 2 is an enlarged top view of the substrate of FIG. 1;

FIG. 3 is a greatly enlarged top view of a sensing system on the substrate of FIG. 2; and

FIG. 4 is an enlarged side sectional view of a sensing system on the substrate of FIG. 2;

FIG. 5 is a top plan view of a region of interest on the platform of FIG. 1 showing four spatially arranged sites with attached cells;

FIG. 6 is a schematic side view of a hydrodynamic cell manipulation system according to another embodiment of the present invention;

FIG. 7 shows hypothetical liquid flow patterns from a tube into a liquid medium;

FIG. 8 shows experimental flow patterns for a dye from a tube into a liquid medium typical for syringe-and-tube based hydrodynamic cell manipulation: FIG. 8A shows typical flow patterns from side view,

FIGS. 8B-D show flow patterns from top view, with fast flow (V(0,0)≈3 cm/s; FIG. 8B), moderate flow (V(0,0) 1 cm/s; FIG. 8C), and slow flow (V(0,0)≈0.12 cm/s; FIG. 8D);

FIG. 9 shows plots of probability of cell attachment at two cell velocities (60 μm/second and 140 μm/second) on three substrates: Si/SiO₂ (FIG. 9A), Si (FIG. 9B), and polyimide (FIG. 9C);

FIG. 10 is a plot of manipulated cell velocity vs. average flow velocity at the outlet of a tube of the manipulator of FIG. 1 on polyimide; and

FIG. 11 shows sequential images of cell movement on a cell interrogator chip using the hydrodynamic cell manipulation system of FIG. 1; and

FIG. 12 provides photomicrographs of preselected cell patterns formed by hydrodynamic cell manipulation using the hydrodynamic cell manipulation system of FIG. 1: FIG. 12A shows a Y-shaped geometry; FIG. 12B shows a collection of densely packed cells in the opening.

### DETAILED DESCRIPTION OF THE PREFERRED EMBODIMENT

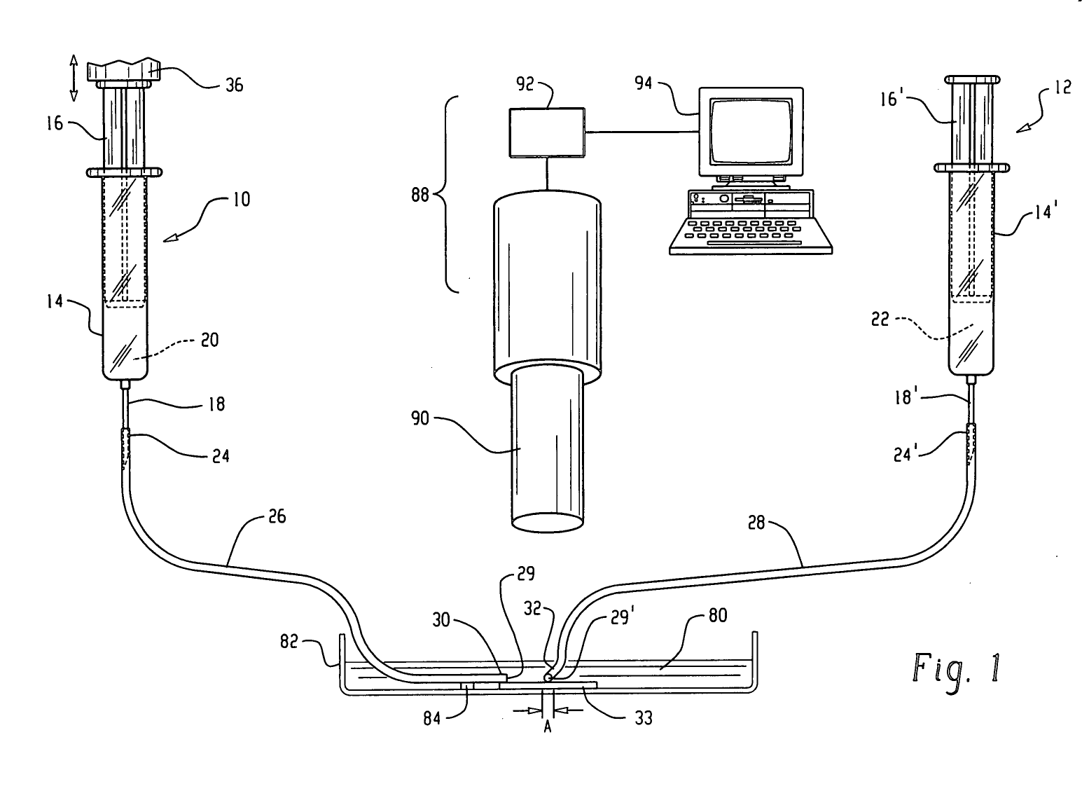

With reference to FIG. 1, a hydrodynamic cell manipulation system is shown. The system allows the controlled navigation of selected living or dead cells, such as human or animal cells, either singly or in small groups. For example a group of less than 50 or less than 10 cells may be readily moved with the system. The system is capable of manual operation, although it is also contemplated that controlled operation be employed. While particular reference is made herein to movement of cells, it will be appreciated that movement of other biological and non-biological objects is also contemplated.

The system includes first and second fluid delivery devices 10, 12, such as graduated syringes. Both syringes may be identical in construction. Each syringe includes a body 14, 14′ which receives a plunger 16, 16′ at one end. The other end of the body has a narrow outlet 18, 18′ in the form of a needle. The body contains a fluid 20, 22, such as a liquid or gas. Both syringes 10, 12 may contain the same fluid, although it is also contemplated that different fluids may be used in each. For example, each syringe may hold about 5-20 cm³ of liquid, e.g., about 10 cm³. In one embodiment, the fluid is a liquid which is not harmful to the cells to be manipulated, such as water or an aqueous solution, such as phosphate buffered saline (PBS) solution.

The narrow outlets 18, 18′ of each syringe are connected to one end 24, 24′ of a hollow tube 26, 28, respectively. The tubes 26, 28 may have an internal diameter of up to a few millimeters for individual cell manipulation purposes, e.g., about 5 mm, more preferably, less than about 3 mm. The internal diameter is preferably larger than that of a cell to be manipulated (diameter of a cell is about 10-30 μg). In one embodiment, the internal diameter is at least about 10 times, and more preferably, at least about 100 times that of a cell. The cell is thus positioned at some distance below the axis of the tube and away from the outlet. Tubes can easily be fabricated with an internal diameter of about 0.5 mm or more. For example, TYGON microbore tube (o. d. =2.0 mm, i. d. =1.4 mm) is attached to the syringe via the needle 18, 18″. In one embodiment, both tubes have the same internal diameter, although it is also contemplated that the internal diameters be different. Outlets 29, 29′ at opposite ends 30, 32 of the tubes 26, 28 are positioned on or adjacent a manipulation platform or cell interrogator chip 33. In one embodiment, the ends 30, 32 of the tubes are physically attached to the platform.

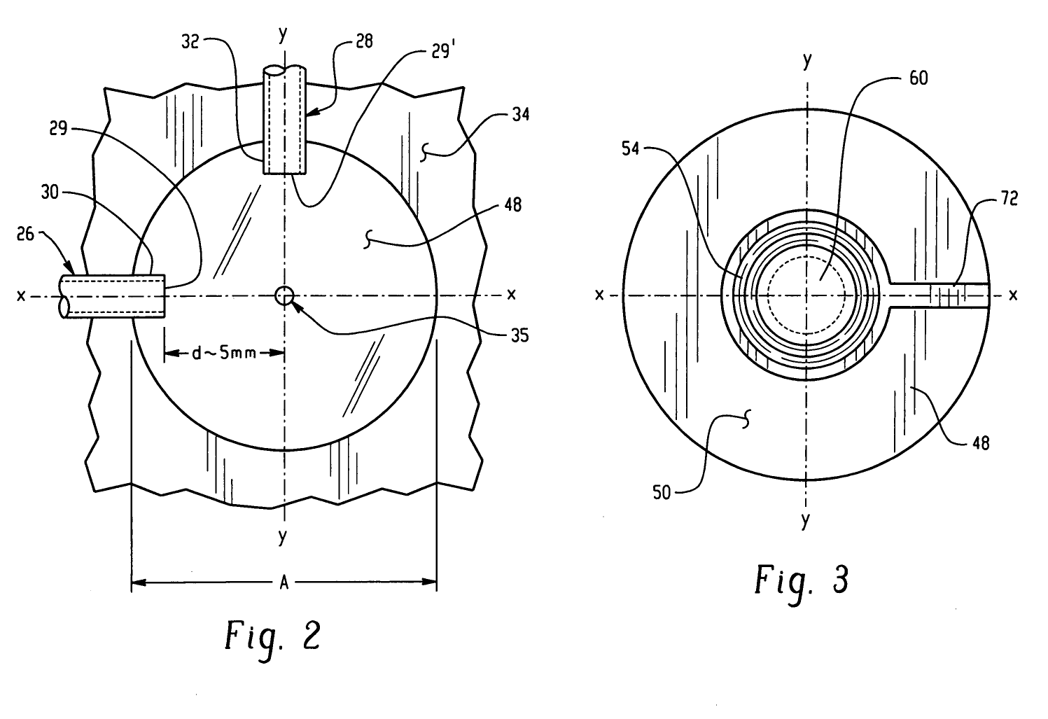

The platform 33 comprises a substrate 34, such as a plate formed from silicon and/or silicon dioxide. As shown in FIG. 2, the ends 30, 32 of the tubes are arranged generally perpendicularly to one another, and preferably in the same plane parallel with the plate, such that liquid from each tube can be directed toward a region of interest 35 of the platform from a distance d of about 0.5-10 mm, about 5 mm in the illustrated embodiment. The flow paths of the liquid from the tubes have respective axes x and y, which travel towards an intercept at or adjacent to the region of interest 35. In one embodiment, the region of interest is positioned at the intercept of the x and y axes defined by the ends 30, 32 of the tubes and the fluid flowing therefrom. The liquid flow from the tubes 26, 28, thus provides x and y axis navigation of a group of cells or a single cell located on the platform.

If the tube outlets 29, 29′ are positioned too close to the region of interest, the tubes themselves may cause optical interference and thus blurring of an image of the region of interest. The minimum distance is thus dependent, to some degree, on the optical path of an imaging system used to view the cells. Additionally, flow patterns at the region of interest 35 can be optimized by selecting an appropriate distance d. The optimum distance d may vary, somewhat, depending on factors such as the size of the cells, viscosity of the fluid, tube diameter, and the like.

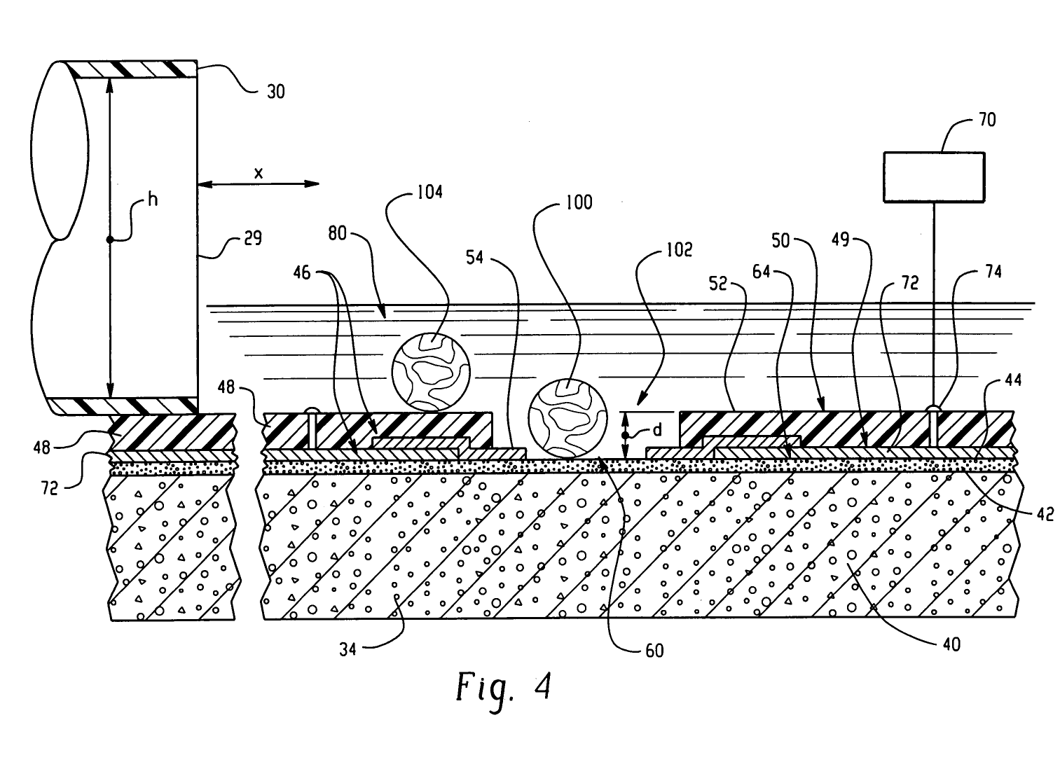

It should be noted that FIG. 2 is an enlarged top plan view of an area A of the substrate, while FIG. 3 shows the central region 35 in enlarged detail. The region of interest 35 typically occupies an area of about 200-1000 μm in diameter, e.g., about 800×600 μm.

The syringe pistons 16, 16′ can be operated manually, for example by pushing on or pulling on the respective piston. Pulling on the piston provides a reverse flow (fluid intake) in a generally opposite direction to that achieved when the syringe piston is depressed. The rate of pushing/pulling can be varied to achieve different cell flow speeds. Alternatively, a motor and drive system can be used to progressively push the piston into the body at a selected rate constant rate or at a variable rate, as described in further detail below. In this way, fluid flows from the tubes 26, 28 can be at the same or at different flow rates. For flow tests, an appropriate weight 36 (e.g., 30 to 150 g) can be attached to the piston to push the piston into the body to achieve reasonably constant delivery speeds.

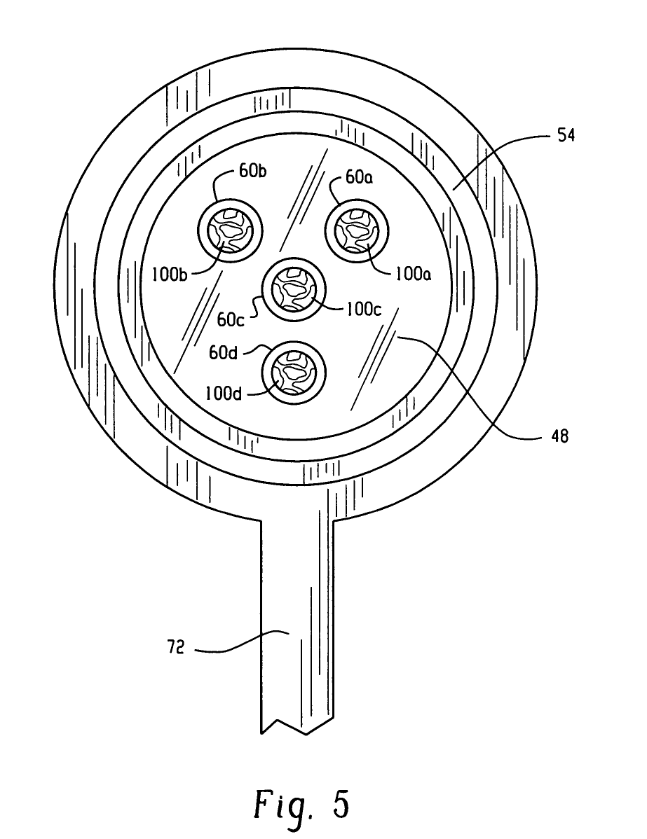

With reference now to FIG. 4, in an illustrated embodiment, the substrate 34 includes a base layer 40 formed from silicon, or other suitable material, and a layer 42 of silicon dioxide (SiO₂) thereon, which may be formed by oxidizing an upper surface of the silicon. The SiO₂ thus forms an upper surface 44 of the substrate. A sensing system 46 can be laid down on the substrate. The substrate base 40 layer acts as a physical support for the various layers supported thereon. In an alternative embodiment, the layer 42 is omitted.

A layer 48 of a hydrophobic material, such as a polyimide, Teflon™, or the like is disposed on an upper surface 49 of the substrate 40 and extends largely across the region of interest 35 to define the majority of an upper surface 50 of the platform 33. A surface 52 of the hydrophobic layer 48 acts to repel cells resulting in no cell attachment or only a weak cell attachment in a selected time period. The polyimide film can be relatively thin, e.g., about 1-5 μm. In one embodiment, the film 48 is about 1.5 μm. The polyimide film is patterned using conventional semiconductor methods, to expose areas which are used for suitable microsensors. In one embodiment, the microsensors include one or more concentric platinum or gold ring electrodes 54 (one in the illustrated embodiment), which are formed on the substrate or otherwise supported thereby. While one electrode is shown, it will be appreciated that fewer or more electrodes may be employed. Additionally, the electrodes may have different configurations from the rings illustrated. In the illustrated embodiment, the electrode consists of a continuous ring. In another embodiment, a split ring configuration is used to provide two electrodes. For electrochemical monitoring, reference and/or counter electrodes (not shown) may be formed on the platform 33 or formed separately and contacted with a liquid medium in which the platform is positioned.

At a site 60 or sites within the polyimide film, the underlying Si/SiO₂ is exposed from the polyimide film to define a hydrophilic area. The site or sites may have a diameter (or average width, if non-circular) of up to about 100 μm, or more. In one embodiment, useful for locating a single cell or a small group of cells, the site is about 20-30 micrometers (μm), although smaller or larger areas may be employed. For example, where it is desired to position only a single cell or a few cells on the site, a diameter of less than 20 μm may be appropriate. The site can thus vary from several microns in diameter, accommodating single small live cells, up to the size of macroscopic cell clusters.

In the illustrated embodiment, a patterned aluminum layer 64 serves as a conductor. The ring platinum electrode(s) 54 make electrical contact to associated electrochemical test instrumentation, such as a potentiostat 70, via buried straight aluminum wires 72 and wire-bondable contact pads 74, exposed on the ends of the aluminum wires (FIG. 4).

The various layers of the platform 33 are laid down by suitable microfabrication techniques, including patterning, etching, and the like.

With reference once more to FIG. 1, the platform 33 and tube ends 30, 32 are covered by a liquid medium 80, which can be the same as the liquid in the syringes, or a different liquid. In the illustrated embodiment, the liquid is the same, e.g., PBS. A Petri dish 82, or other suitable container, holds the liquid medium 80. The depth of the liquid medium above the platform can be relatively shallow, such as about 1-5 mm, such that flow from the tubes is relatively laminar. The depth of the liquid medium is preferably greater than the external diameter of the tubes 26, 28 such that all the liquid from the tube flows into the liquid medium 80. The tubes 26, 28 may be attached to the container, adjacent their ends 30, 32, as illustrated in FIG. 1, with an adhesive 84 or other suitable fixing member. This allows the platform 33 to be removed and replaced without the need to remove the tubes and reposition them orthogonally. Alternatively, a lifting device (not shown) can be used to lower the tube ends 30, 32 into position on the platform 33 and raise them when it is desired to remove the platform.

The movement of cells within the region of interest 35 can be observed as magnified images with an imaging system 88 including a microscope 90, which may be monitored by a CCD video camera 92 and acquired and stored by a personal computer 94 with a video capture board, as illustrated schematically on FIG. 1. While these are shown as being positioned above the platform, it is also contemplated that the imaging system be positioned below the platform where the substrate is formed from a transparent material.

Prior to using the system to position cells, flow rates may be observed by use of a tracer, such as a dye. A small amount of a visually detectable dye is located in the liquid medium 80, at or adjacent one of the tube outlets 29, 29′, intermediate the tube outlet and region of interest 35. The movement of the dye during flow from the syringes 10, 12 is observed, preferably in two axes, such as from above and from the side of the platform 33. The movement can be recorded as still frames using the CCD camera 92 and the dye flow over time is thus readily apparent. By repeating the experiment at different syringe pushing rates, the optimum pushing rates can be determined for the system, which allow cells to be easily navigated in a short period of time.

Due to the properties of viscous flow, the macroscopic flow at the height h of the tube outlet very rapidly diminishes to very low speeds at the vicinity of the substrate where settled cells are located. A relatively coarse system realized by pushing and/or pulling the syringes, providing a few millimeters/second flow rates (e.g., 0-30 mm/second, in one embodiment, less than 20 mm/second, in another embodiment, at least 1 mm/second, in another embodiment, 2-10 mm/second, in yet another embodiment, less than 5 mm/second) at the tube ends 30, 32, allows fine and precise movement of actual cells on the platform 33. The average flow velocity of the liquid at the tube outlet Vₐᵥ is greater than that of the cell velocity V_{cell}. The exact ratio of Vₐᵥ/V_{cell} depends, in part, on the ratio of the tube internal diameter to the diameter of the cell. In one embodiment Vₐᵥ is at least ten times that of V_{cell} and can be up to about 10,000 times V_{cell}. In one embodiment, the average flow velocity of the liquid at the tube outlet Vₐᵥ is at least about 100 times that of the manipulated cell velocity V_{cell}. In another embodiment, the average flow velocity of the liquid at the tube outlet Vₐᵥ is less than about 1000 times that of the manipulated cell velocity. In one embodiment, the suspended cells move at a speed of about 1 to 120 micrometers (μm) per second.

For a navigation step, a useful cell velocity V_{cell} is about 1 to 20 micrometers (μm) per second. Higher cell velocity V_{cell} and correspondingly higher average flow velocity Vₐᵥ can be used in a cleaning stage where unwanted cells are removed from the region of interest (e.g., 5-100 μm/second, and in one embodiment, 10-50 μm/second). The optimum cell flow rates (cell velocities) will depend, to some degree, on whether human or computer-controlled manipulation of the syringes is used.

The use of two orthogonal tubes makes any cell trajectory feasible by pushing and/or pulling the syringe pistons, 16, 16′ at a suitable rate. It will be appreciated that although the ends 30, 32 of the tubes are described as being aligned with x and y axes which are orthogonal (i.e., at 90° to one another), it should be appreciated that manipulation may be achieved where the tubes are angularly spaced at a different angle, such as from 40-130°, more preferably, from 60-120°. Experiments using a 56° angular spacing between tubes showed manipulation is feasible in under one minute even when tubes are not orthogonal.

Once optimum flow rates are determined, the system can be used to rapidly and accurately position cells 100 on the attachment cites 60 (FIG. 4). The cells may be added as a suspension to the liquid medium 80. For example, a selected amount (e.g., about 5 to 100 μL) of a suspension of cells at a known cell concentration (about 10⁻⁵ cells/mL) is added to the liquid medium 80 using a suitable delivery device, such as a pipettor. The cells are suspended in a liquid which can be the same as the liquid medium 80 or a different liquid.

It will be appreciated that FIG. 4 is not shown to scale, to accentuate the features being discussed. In practice, the cells 100, when located on the site 60 extend substantially above the polyimide layer 48, rather than being largely disposed within an opening or shallow well 102, as shown. The depth d of the opening 102 can be generally less than about 5 μm, in one embodiment, about 1-2 μm. Thus, the topological depth of the opening 102 has little or no influence on the trapping of the cell 100, which is due primarily or exclusively to the hydrophilic nature of the site 60.

After the cell suspension has been added to the liquid medium, the cells 100 are allowed to settle on the platform 33, which takes a few seconds, typically about 10-20 seconds (settling step). This can be observed by tuning the focus plane of the microscope to just above the substrate 34. After most of the cells have settled on the platform, cell navigation is commenced. A selected individual target cell, which is preferably located within the region of interest, can be navigated to a selected hydrophilic site 60 at an Si/SiO₂ opening by controlling (pushing and/or pulling at an appropriate speed) the x and y syringes 10, 12. This may be achieved in several discrete steps in which first one syringe, then the other syringe, is controlled to move the cell a short distance. Alternatively, both syringes may be operated concurrently. This navigation step can be readily achieved in about one minute.

Once the cell or cells have been navigated to the selected site or sites 60, the cells are allowed a period of time for attachment to the site 60 (attachment or trapping step). Five minutes has been found adequate for this attachment period, although shorter or longer times may be used, depending on the type of cells, substrate materials, and other factors.

Unwanted cells 104 (FIG. 4), which are substantially unattached or only weakly attached, are then removed from the substrate (cleaning step). These cells, which are located on the hydrophobic layer 48, are readily removed by a cleaning process, without dislodging the attached cells 100 at the Si/SiO₂ sites 60. For example, the syringes 10, 12 are controlled to provide a sufficient flow rate to move the surrounding cells to a suitable distance from the site 60. This distance is selected to be sufficient such that the non-attached cells 104 will not interfere with any subsequent tests on the attached cells. For example a cell velocity V_{CELL} of about 5-50 μm/second is appropriate for this step. The velocity is chosen to be such as to remove the unwanted cells while leaving all or substantially all attached cells on the site 60.

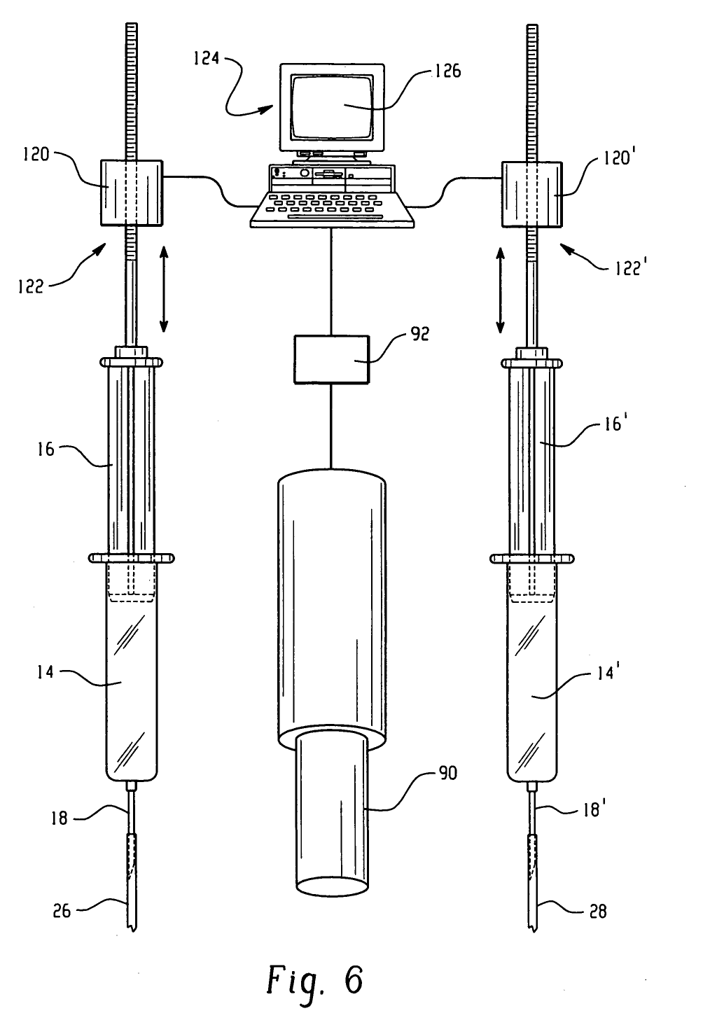

It will be appreciated that the number of cells on any particular site 60 can be accurately controlled using the methods described. For example, one cell or a group of cells may be navigated to the site 60. Additionally, spatial arrangements of cells can be achieved by positioning a number of sites at selected distances and directions from each other. By way of example, FIG. 5 illustrates a Y-shaped arrangement of sites 60a, 60b, 60c, 60d. Each of the sites may be sized to receive a single cell 100a, 100b, 100c, 100d.

A pattern, such as that shown in FIG. 5, can also be achieved by manipulating several cells into the desired locations on a single attachment site 60. For example, a single cell is navigated to the site, trapped, then unwanted cells removed. By repeating this single cell navigating, trapping, and cleaning procedure several times, a variety of pre-selected desired formations of small, clearly defined cell clusters can be obtained.

The system takes advantage of the difference in the rate of cell attachment to the hydrophobic portions 48 of the platform 33 as compared with the rate of attachment to hydrophilic portions (sites 60). Although not fully understood, this attachment is primarily by physical rather than biological means, as it takes place rapidly, generally within about one to five minutes, depending on the type of substrate and other factors, such as the size of the cell. For a silicon/silicon dioxide substrate, the probability of a cell attaching in one minute is close to 1 (i.e., substantially all cells will attach in this time), even at relatively high cell removal velocities of 60 μm/second. On a silicon substrate, the probability in one minute is closer to 0.7 at these velocities, with a probability of close to 1 being reached after about 4 minutes. This is still substantially higher than for polyimide, where the probability is about 0.4 after 1 minute.

It will be appreciated that although the area 52 of hydrophobic portion 48 is referred to as hydrophobic and sites 60 are described as hydrophilic, the difference in the strength of attachment may also be achieved by having two hydrophilic materials or two hydrophobic materials, one of which (area 52) is less hydrophilic than the other. The differences in rates of attachment of the cells due to the hydrophobic or hydrophilic nature of the two materials can be studied under various cell flow speeds and the probability of cells to stay attached on these areas 52, 60 can be determined. Based on the studies, an appropriate attachment period can be determined for the materials selected.

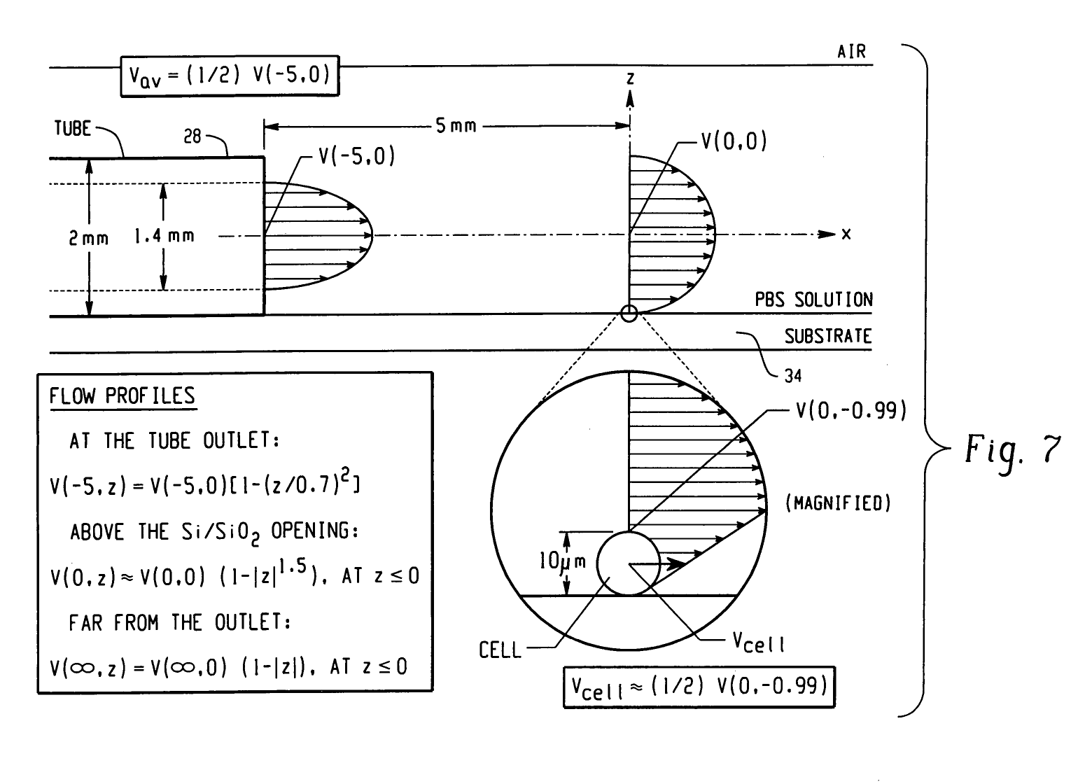

FIG. 6 illustrates an alternative fluid delivery system which includes motors 120, 120′ and drive systems 122, 122′ for progressively pushing/pulling the piston 16, 16′of each syringe into/out of the body 14, 14′ at a selected rate, which may be a constant rate or at a variable rate. In this way, fluid flows from the tube outlets 29, 29′can be at the same or at different flow rates. The motors 120, 120′ may be controlled by a computer 124. Once the desired cell(s) and the region of interest 35 are selected on the computer screen 126, computer programs including image processing can be used for feedback. Optimum trajectories can then be calculated by the computer that account for other present cells and even obstructions. Flow speed can also be optimized. Instead of discrete x- and y-steps, trajectories of any angle can be produced, including curved ones to circumnavigate obstacles.

In another embodiment (not shown), a single syringe similar to syringe 10 or 12 is used. The syringe is connected with a valve connected with both the first and second tubes. By shifting the valve position, the single syringe is alternately fluidly connected with the first or second tube.

Once the unwanted cells are removed, the platform 33 may remain in the liquid medium 80 for undertaking studies on the attached cells, or be removed from the liquid medium 80 into a separate medium. The study can employ electrochemical or optical means. For example, studies of drug uptake and/or efflux of the cells may be undertaken. Drug uptake studies may include delivering a drug or other chemical of interest to the liquid medium or immersing the platform in a solution containing the drug, and studying uptake by the cells. Dug efflux (drug egress) studies may be carried out in a similar manner, but including further steps of removing the platform from the drug containing liquid after uptake has occurred and optionally washing the platform with a fresh, drug-free liquid to remove excess drug from the exterior of the cells. Efflux is then detected after placing the platform with the drug-containing cells in a fresh, drug-free liquid.

Alternatively, for efflux studies, the cells can be treated with a drug prior to micromanipulation. Such studies benefit from the rapid positioning of the cells because efflux begins as soon as the drug-containing cells are transferred to a drug-free liquid.

For example, studies can be made of detected changes in current or voltage across the electrode(s) 54 when a voltage or current is applied using the potentiostat 70, as illustrated in FIG. 4, or other suitable electrochemical monitoring equipment. The electrochemical monitoring system comprising the electrodes and monitoring equipment shown may be used, for example, for oxygen monitoring and other amperometric applications. The electrode(s) respond to changes in the liquid surrounding the cells and thus are positioned close enough to the cell attachment site 60 to measure the changes, e.g., within about 100 μm of the site.

Optical studies can also be carried out. In the case of an optically detectable chemical, such a drug labeled with a dye or fluorescent tag, or a drug which is capable of reacting with a second chemical to produce fluorescence, detection of the drug influx/efflux may be made by a microscope and camera arrangement, similar to that discussed with respect to FIG. 1. It will be appreciated that for such studies, the platform 33 need not have the electrode(s) 54 and contact wire 72 formed thereon. Combinations of monitoring studies can be carried out, such as a combination of optical and electrochemical monitoring.

Measurements of drug influx and efflux have particular application in the study of drugs for treatment of cancer. Certain cancers are known to develop multi-drug resistance, which leads to changes in the drug uptake and/or efflux. These changes can be studied on isolated cells, small groups of cells or cells which are spatially positioned at selected distances and orientations to one another, as achieved by the above described micromanipulation methods.

Correlation between average flow velocity Vₐᵥ at the exit 29, 29′ of the tube and induced cell velocity V_{Cell} can be determined by pushing a syringe at several different constant rates. At each rate, the distance that a settled cell travels on the hydrophobic polyimide layer 48 during the time of observation is measured under the microscope, and cell travel speed (V_{cell}) is calculated. The volume pushed out from the tube during this interval is also read from the syringe measure, to calculate the volumetric flow rate (Q) and the average flow velocity Vₐᵥ at the outlet of the tube.

The micromanipulation technique is gentler on the cells than conventional mechanical manipulation techniques. It allows precise navigation of the cells onto a desired site, such as a patterned biomicromechechanical system (BIOMEM) chip or under an optical detection system, such as a microscope.

One method of forming the platform includes fabricating structures on a polished side of a prime grade (100) Si wafer, e.g., about 50-200 mm in diameter. The process begins with a standard cleaning of the wafer in order to remove ionic and organic contaminants on the wafer surfaces. Following the cleaning, an SiO₂ film is grown by thermal oxidation on the surface of the Si wafer of about 1-5 μm-in thickness. The oxidizing environment can consist of a mixture of O₂ and H₂, and can be performed at atmospheric pressure at a temperature of about 1000° C. to about 1300° C. After oxidation, an aluminum film is sputter deposited on the SiO₂ surface and may be about 2000-10,000 Å thick. This film is then patterned into metal interconnects using conventional photoresist-based photolithography followed by Al etching in a commercially available aqueous Al etchant. After etching, the photoresist is removed using a metal-safe photoresist stripper.

At this stage, the Pt (or other electrode material) electrodes are fabricated on the wafers. This may be achieved by spin coating with a photoresist at about 1-2 μm and patterning the photoresist to define molds for the Pt electrodes. After photolithography, a film of platinum is sputter deposited on the wafer at about 1500-3000 Å thickness. To improve adhesion of the platinum, an adhesion layer of about 100 Å thickness formed from Ti or other suitable adhesion material, may be deposited on the wafer prior to deposition of Pt. The Ti and Pt deposition can be carried out in the same chamber to avoid formation of an interface oxide between the Pt and Ti layers.

Following electrode patterning, the wafer is ready for polyimide processing using a suitable polyimide, such as HD Microsystems' PI 2616. Each wafer can be first coated with an adhesion promoter, such as an HD Microsystems' VM 652 adhesion promoter, baked (e.g., for 15 min at 90° C.), then coated with a thin layer of polyimide using the spin coating tools normally used for photoresist processing. The thickness for the polyimide film can be about 1-3 μm before curing and about 0.5-2 μm after curing. Curing can be performed in an atmospheric furnace at about 300-600° C. in an inert atmosphere, such as nitrogen, for a suitable time, such as about 120 minutes at 400° C. After curing, the polyimide film is ready for patterning.

Each wafer is first spin coated with a film of photoresist, after which the photoresist is patterned using standard lithography techniques. The polyimide film is then etched in an O₂ plasma using an SiO₂ plasma etch tool configured for O₂-based plasmas. The plasma can consist of a gaseous mixture of O₂, a fluorocarbon, such as C₂F₆, and an inert gas, such as He. Under low vacuum conditions, the polyimide etch rate is about 3700 Å/min. Much of the photoresist is etched during the polyimide etch process, but what remains is simply removed by rinsing in a suitable solvent, such as acetone. The polyimide coating is patterned in such a way that the Al metallization is completely overcoated except at the contact pads, leaving only the Pt electrodes and associated regions 60 of the SiO₂ underlayer exposed. At this point, the wafers are inspected, diced into chips, and otherwise made available for testing.

Where the platform 33 is not to be used for electrochemical measurements, the steps of forming the aluminum layer and platinum electrodes can be eliminated.

Optionally, in addition to or in place of an electrochemical measuring system, optical sensors may be incorporated into the platform 33. One or more optical sensors, such as those for detection of potassium, calcium, magnesium, hydrogen ions, or glucose may be supported on or embedded in the substrate, for example in the form of capsules having a membrane which is permeable to the ion or species of interest to be measured. In one embodiment, a plurality of such sensors are arranged in a ring around the site and exposed at the surface of the platform through the polyimide layer. The sensors exhibit a change in an optical property, such as color or fluorescence, when the local liquid medium experiences a change in the concentration of the ion or species to be detected. Thus, where efflux of an ion from the cell occurs, the localized concentration is increased and is recognized by the sensor as a change in color, fluorescence, or the like. The color changes can be observed from above (or below) the platform using the imaging system 88 or other suitable equipment.

It has been found that the geometry of the cell culture (spatial distribution of cells) under study exerts a major, hitherto largely neglected, influence on biological transport characteristics. Doxorubicin efflux, for example, from loose populations of suspended cells from an almost confluent monolayer of the same cells, and from single cells differed greatly despite the otherwise identical experimental conditions. For quantitatively assessment of cellular transport (or communication) in vitro, the precisely defined cell or cells achieved with the micromanipulation system of the present invention are particularly effective.

Cell patterns are predefined by a corresponding pattern of hydrophilic sites 60 for cell attachment that are surrounded by hydrophobic surfaces 52 that tend to repel cells. Using microfabrication techniques, cell sites can be varied from several microns in diameter for accommodating single live cells, to larger sites for small cell clusters, up to macroscopic cell layers. The hydrophobic coating 48 outside the cell attachment 60 sites can have virtually zero thickness, thereby eliminating any geometrical interference on cellular transport among cells.

The simplicity of the syringe-and-tube based micromanipulation technique also finds application in the areas of gene transfection, drug injection, and other cellular engineering applications. Hydrophilic/hydrophobic patterns fabricated with MEMS technology on the micron scale can be used even in the five minutes time range for selective and lasting cell attachment to predetermined sites. The system also allows dynamic studies on cellular transport and communication using BioMEMS technology. This is because BioMEMS chips can also accommodate precisely aligned sensors and cells. Optical microscopies are readily available for assessing different aspects of transport at cell patterns on a cell interrogator, especially one using a transparent substrate. Patterning of suitable MEMS-based microsensors at and around cell sites on such cell interrogator chips can be used to directly monitor local concentrations and thus, (indirectly) assess chemical fluxes at the cells. A combination of such local sensors and optical microscopies can then cover a broad range of variables (electrical, chemical, and even mechanical) that, in principle, can be monitored simultaneously at such cell preparations.

A further unique characteristic of the cell interrogator chip is that micro-electromechanical actuators based on piezoelectric microstructures can be added to achieve local cell stimulation, or drug delivery into selected cells using similar principles for delivery as those applied in diffusional microburettes. Thus, individual cells or cell clusters can be addressed (“interrogated”) in later designs not only in terms of sensing but also, in terms of electrical, mechanical, and/or chemical stimulation.

The cell interrogator chips 33 can also provide exceptionally good quantitative reliability. This aspect can solve one of the major problems in pharmacological research and development: the need for high statistical quality of the data. This is achieved because a large number of identical experiments can be performed on the same chip simultaneously at many identical sites.

In one embodiment, the Pt electrode or other sensors can be used themselves to produce dielectrophoretic forces to pull cells towards the electrode. It is possible to completely attach them this way to sites at an electrochemical sensor, or may be used simply in the preselection of cells, i.e., to get cells close to sites and then use hydrodynamic manipulation for fine tuning.

While the invention has been described with reference to the positioning of cells, it will be appreciated that smaller entities, such as parts of cells (e.g., genes, large proteins) may be manipulated, as well as aggregated masses of cells, such a section from a tumor.

Theoretical Considerations

While not intended to limit the invention, the following theoretical considerations may provide a possible explanation for the micromanipulation process.

Flow Patterns

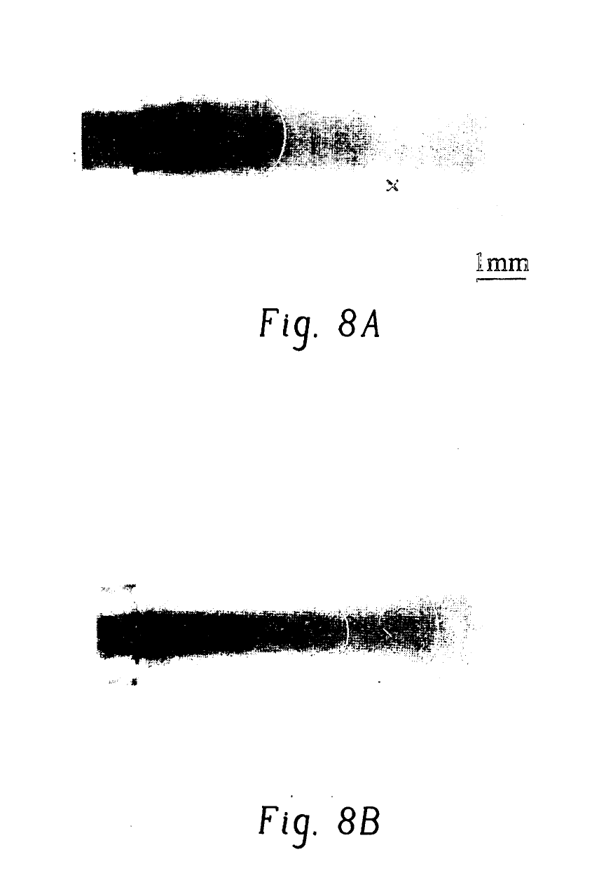

Schematics of flow patterns in the syringe-and-tube system, and the adopted coordinate system are shown in FIG. 7. The side view of hypothesized flow patterns is shown, with the x-z coordinate system defined as follows. The x-axis is along the axis of the x-tube 26, with x being zero above the center of the Si/SiO₂ opening 60 on the chip and pointing away from the tube. The y-axis (not shown here but shown in FIG. 2) coincides with the axis of the y-tube 28, similar to the definition of the x-axis. The manipulated cells are located about 5 mm away from both outlets, the x outlet being thus at x=−5 mm. The z-axis is vertical (perpendicular to the surface of the substrate 34) directed upwards from the axis of the x-tube where z=0. The surface of the substrate is thus at about z=−1 mm (based on the o.d. of the tube being used). V(α,β) represents flow velocity at x=α mm and z=β mm. Vₐᵥ and V_{cell} are the average flow velocity at the outlet of the tube and the manipulated cell velocity on polyimide, respectively. Cells are hypothesized here as spheres of 10 μm diameter.

Because the distance of the region of interest (ROI) 35 including the Si/SiO₂ opening 60 is about 5 mm from the tube outlet 29, 29′and the size of the ROI is in the order of 800×600 μm, the manipulated cells can be considered to be all in the axial vertical (x-z) plane. Thus, it is sufficient to estimate flow patterns in that plane to derive velocities of cells settled on the substrate, meaning that flow velocity at any location of interest can be represented as V(x,z). The manipulated cell velocity (V_{cell}) is approximated as one half of the flow at the top of a settled cell, based on the assumption that cells are rolling with neither slipping nor sticking on the chip.

Flow in every studied case was found to be essentially laminar everywhere in the flow stream. The Reynolds number is already very small inside the tube in practical cell manipulations: Re ˜7 for the typical average flow velocity in the tube, Vₐᵥ ˜1 cm/s, corresponding to a volumetric flow rate, Q˜15 μL/s. The flow then expands and changes symmetry upon exit. While axial symmetry is then lost in three dimensions, it is preserved in the horizontal plane. This flow expansion will increase the Reynolds number outside the tube to some extent. The flow, however, is expected to remain laminar because of the shallow solution layer (˜3 mm) and low flow rates used.

Stationary laminar flow produces parabolic flow patterns in a tube (FIG. 7). On the other hand, shear flow above a plane in steady state is linear in the axial vertical plane sufficiently far from the outlet. Flow at the ROI (5 mm from the outlet) can therefore be assumed to be one between parabolic and linear, that may be approximated as V(0,z) V(0,0)(1−|z|^{1.5}) at z≦0.

Cell Attachment

The probability of acute (physical) cell attachment, P, is defined by the following relationship:
P=N_{attached}/Nₜₒₜₐₗ (1)
where Nₜₒₜₐₗ is the total number of cells observed in the ROI before a cell attachment experiment, and N_{attached} is the number of cells that remained attached after the set flow-speed (V_{cell}) was applied. The experimentally obtained relationships between the cell-substrate contact time, t, and the probability for cells to stay attached, P, can be compared with theoretical data. An exponential time course could be fit reasonably well to each data set:
   P(t)=1−e^{·t/τ} (2)

Where t is time and τ is the time constant of apparent attachment kinetics. The pre-exponential factor is 1 (or 100%) since all cells will ultimately become attached after allowing sufficient time.

To compare quantitatively the attachment tendencies for the different substrates and flow rates, each data set is least squares fitted with this function. Due to normalization, the time constant, τ, can be used alone to characterize attachment kinetics. Smaller τ values mean stronger and faster attachment. For example, the time constants for cells on Si/SiO₂ were about five times less than those on polyimide, meaning that the former is much more suitable for cell attachment sites.

Without intending to limit the scope of the invention, the following examples describe fabrication and operation of a hydrodynamic micromanipulation system and the effectiveness of the micromanipulation technique.

### EXAMPLES

All chemicals used in the experiments are of analytical grade. Quartz distilled water (18 MΩcm²) is used to prepare all solutions.

### Example 1

### Platform Fabrication

Substrate structures are fabricated on 100 mm-diameter, single side polished, prime grade (100) Si wafers. For these devices, all pertinent processing is performed on the polished side of each wafer. The process begins with a standard RCA cleaning of the wafers in order to remove ionic and organic contaminants on the wafer surfaces. Immediately following the RCA clean, a 1.5 μm-thick, SiO₂ film is grown by thermal oxidation on the surface of each Si wafer. The oxidizing environment consists of a mixture of O₂ and H₂, and is performed at atmospheric pressure at a temperature of 1100° C. After oxidation, a 5000 Å thick aluminum film is sputter deposited on the SiO₂ surface. This film is then patterned into metal interconnects using conventional photoresist-based photolithography followed by Al etching in a commercially available aqueous Al etchant. After etching, the photoresist is removed using a metal-safe photoresist stripper. At this stage, the Pt electrodes are fabricated on the wafers.

Following electrode patterning, the wafers are ready for polyimide processing using a polyimide (HD Microsystems PI 2616). Each wafer is first coated with an adhesion promoter (HD Microsystems VM 652), baked for 15 min at 90° C., then coated with a thin layer of polyimide using the spin coating tools normally used for photoresist processing. The target thickness for the polyimide film is 1.7 μm before curing and about 1.5 μm after curing. Curing is performed in an atmospheric furnace at 400° C. in nitrogen for 120 min, with temperature ramps from 150 to 400° C. at 3° C./min prior to curing and from 400 to 500° C. at 3° C./min after curing. After curing, the polyimide films are ready for patterning.

Each wafer is first spin coated with a 10 μm-thick film of AZ 9260 photoresist, after which the photoresist is patterned using standard lithography techniques. The polyimide films are then etched in an O₂ plasma using a Tegal 803 SiO₂ plasma etch tool specially configured (but not optimized) for O₂-based plasmas. The plasma consists of the following gaseous mixture: 41% O₂, 8% C₂F₆, and 51% He. The forward power is about 189 W and the chamber pressure is about 2.8 Torr. Under these conditions, the polyimide etch rate is about 3700 Å/min. Much of the photoresist is etched during the polyimide etch process, but what remains is simply removed by rinsing in acetone. The polyimide coating is patterned in such a way that the Al metallization is completely overcoated except at the contact pads, leaving only the Pt electrodes and associated regions of the SiO₂ underlayer exposed. At this point, the wafers are inspected, diced into chips, and otherwise made available for testing.

Where the chip 33 is not to be used for electrochemical measurements, the steps of forming the aluminum layer and platinum electrodes can be eliminated.

### Example 2

### Assembly of a Micromanipulator System

A platform prepared as for Example 1 is placed under PBS solution on the bottom of a Petri dish (60×15 mm polystyrene). An appropriate weight (30 to 150 g) is attached to the tail of a 10 cc syringe so that the piston is pushed down by gravity and balanced by friction to achieve reasonably constant speed. One end of a TYGON microbore tube (o. d. =2.0 mm, i. d. =1.4 mm) is attached to the syringe via a needle (16_{G} 1, Becton Dickinson, Rutherford, N.J.). The other end of the tube is placed horizontally on the platform under the PBS, facing the region of interest from 5 mm away. The syringe-connected tubes are aligned in the x and y directions, respectively, so that they can manipulate cells towards the Si/SiO₂ opening 60, which is surrounded by a suitable microsensor or sensors such as a Pt micro-ring electrode. The outer sensor edge and the connecting Al wire are embedded in a continuous polyimide surface that rejects cells due to its relative hydrophobicity. The hydrophilic Si/SiO₂ opening is exposed from the polyimide coating so that cells can attach there.

A reflectance microscope (Leitz Secolux 6″×6″ Inspection Microscope, Heerbrugg, Switzerland) with a 10× objective lens (NA 0.22, Leitz, Heerbrugg, Switzerland) is used to observe the used to observe the magnified images of cells around the target attachment site. Images are monitored by a CCD camera (Bausch & Lomb TU Camera, JE 3010A, Torrance, Calif.), and recorded by a VCR, and acquired and stored by a PC with a video capture board.

### Example 3

### Flow Pattern Tests

To test the syringe and tube manipulation system of Example 2, a fresh platform is placed under PBS solution in a polystyrene Petri dish (Becton Dickinson, 60×15 mm). Two 10 cm³ (Becton Dickinson) are connected with Tygon™ microbore tube (o.d. +2.0 mm, i.d. =1,4 mm) via needles (Becton Dickinson 16_{G} 1) for navigation in the x and y axes, respectively. Ends of the tubes are positioned at about 5 mm from a region of interest 35.

To monitor flow patterns, a blue-colored dye (Evans blue, Allied Chemical) is employed as a flow marker. A video zoom microscope (Edmund Industrial Optics VZM 300 color system) is used to observe and record flows. Visualized flows are stored as movies (about 5 frames/second) and converted to still images with time labels by the personal computer.

Successive images are generated for the movement of the dye at different syringe pushing rates, monitored from both the top and the side of the platform. Over the short time span of the experiment, it is anticipated that diffusion does not contribute significantly to the observed dye expansion. (With a flow rate F of about 10⁻⁶ cm²/second, diffusional expansion is about (Ft)^{1/2}=10 μm in 1 second).

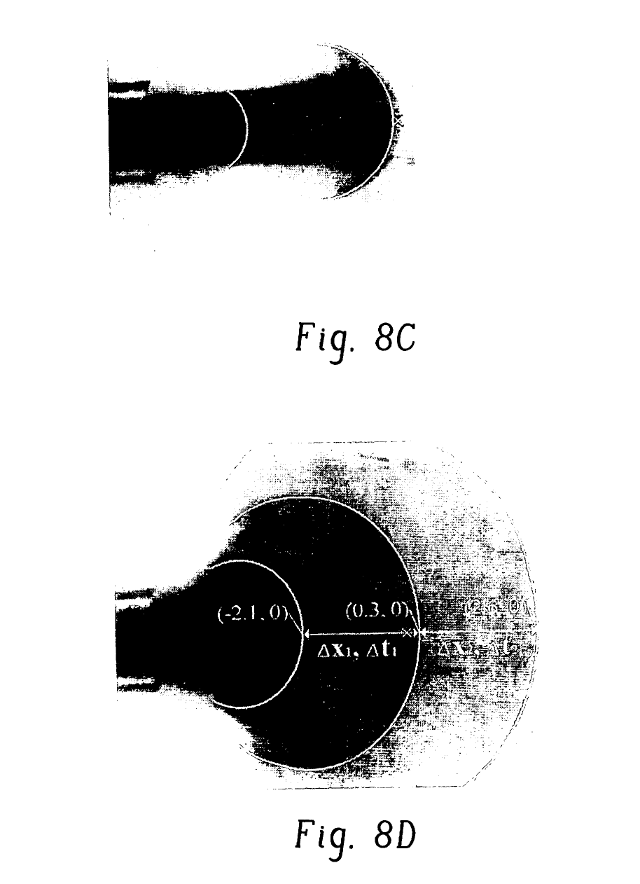

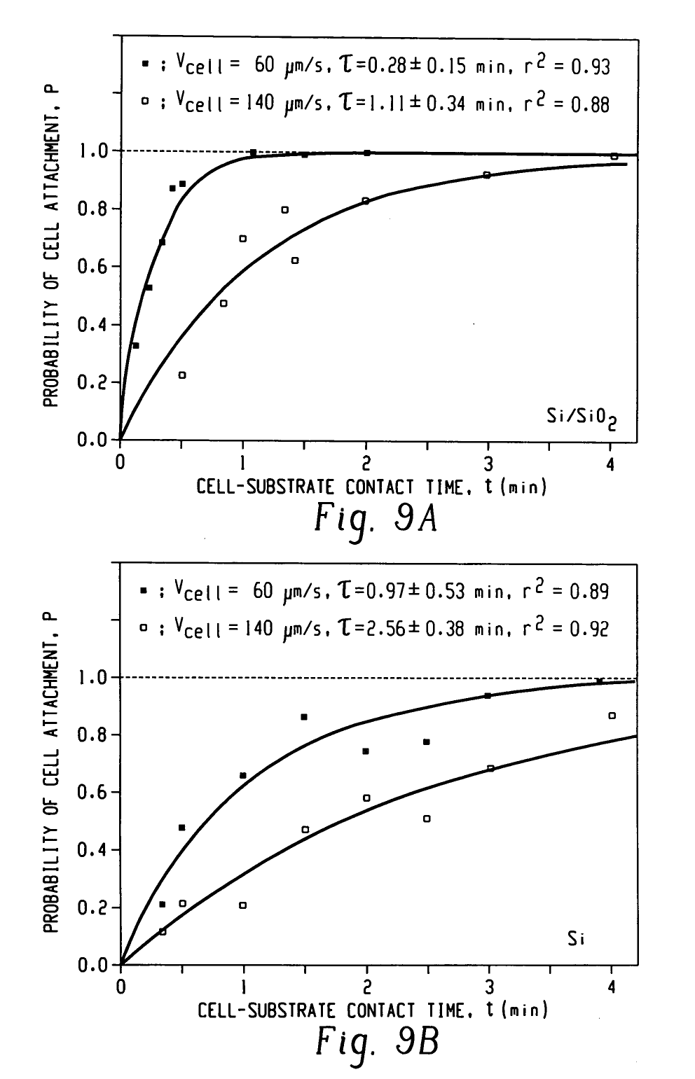

Experimental flow patterns typical for syringe-and-tube based hydrodynamic cell manipulation are shown in FIG. 8. Three still pictures (snapshots) at different instances were superimposed to make each panel, resulting in earlier flow patterns becoming darker in the gray-scale. The front contour of each snapshot is emphasized with white lines. FIG. 8A shows typical flow patterns from a side view, that were found similar for the different flow velocities used. FIG. 8B-D show flow patterns from top view, with fast flow (V(0,0)=3 cm/s; FIG. 8B), moderate flow (V(0,0) 1 cm/s; FIG. 8C), and slow flow (V(0,0)≈0.12 cm/s; FIG. 8D). Cross marks (x) represent the approximate position of the Si/SiO₂ opening 60 (5 mm distance from the outlet 29 of the x-tube 26).

While the vertical components of representative flow patterns, observed from the side, did not significantly change with increasing flow velocity (FIG. 8A), the horizontal patterns obtained from the top view strongly depended on absolute velocity (FIG. 8B-D). From a narrow jet (FIG. 8B) at relatively high speeds, through moderate broadening (FIG. 8C) and axisymmetric fanning (FIG. 8D), to almost circular expansion around the tube outlet at very low speeds (not shown), a variety of patterns are feasible dependent on syringe moving rate. This means that a wide range of cell travel velocities can be produced within the same system.

### Example 4

### Cell Attachment Study

Twenty μL of macrophage cells (approximately 10⁵ cells/mL) are dispersed around the region of interest on a substrate by a micro-pipette. After waiting for all cells to settle down on the substrate (10-20 s), which is observed by tuning the microscope focus to just above the substrate, the experiment is initiated. This consists of assessing the relationship between the cell-substrate contact time, and the probability for the cells to stay attached, at two flow speeds. Cell-substrate contact time is measured by a manual stop watch. The time period of contact is reset to zero by forcing all cells seen in the region to detach. This is achieved by pushing and pulling the syringe inducing relatively fast flow (volume flow rate ˜30 μL/s, corresponding to cell flow speed on the substrate, V_{cell}, ˜80 μm/s), and then waiting for the cells to re-settle (typically, ˜10 s). After the desired cell-substrate contact time, one of two levels of cell flow speed (V_{cell}) is applied by controlling the weight on the syringe. The speed at which the syringe piston goes down after a weight is placed on the piston reaches a steady speed after a short acceleration period due to second order dynamics of a typical syringe (friction is proportional to the first derivative of displacement while the gravitational force is related to the respective second derivative). Flow speed is then monitored by observing the movement of cells after they detached from the substrate. The velocities, V_{cell}, were 60 μm/s and 140 μm/s.

The flow speed of detached cells and the number of cells that remained attached are finally determined by monitoring VCR recordings made during the experiments in slow playing mode. The probability of acute cell attachment is then calculated as described above, with the total number of cells evaluated in one experiment being approximately 100. This is achieved by repeating the same procedure in the same setup two to three times to ensure sufficient statistical sampling. About 30 to 70 cells can be observed during any one procedure. Flow patterns in the central region 35 are virtually homogeneous, because of its small size (800×600 μm) compared to the entire flow field (expanding from the tube of i.d. =1.4 mm at 5 mm distance). Also, the flow being laminar, irregularities on a microscopic scale are highly unlikely. The attachment to different materials (silicon, Si/SiO₂, polyimide) is studied.

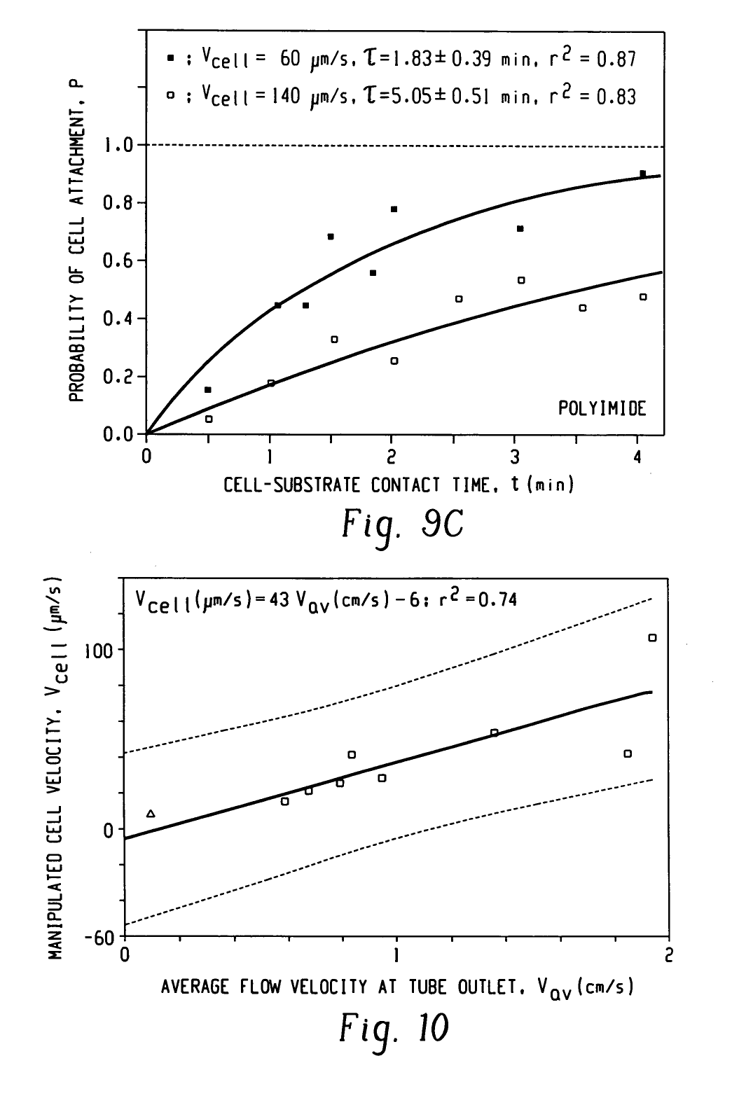

The experimentally-obtained relationships between the cell-substrate contact time, t, and the probability for cells to stay attached, P, are shown in FIG. 9. FIGS. 9A, 9B and 9C show probabilities measured at cell velocities of 60 μm/second (filled squares) and 140 μm/second (open squares) on three different substrates, Si/SiO₂, Si, and polyimide, respectively, over a time period of four minutes.

An exponential time course, as described above, could be fit reasonably well to each data set:
P(t)=1−e^{·t/τ} (2)

To compare quantitatively the attachment tendencies for the different substrates and flow rates, each data set was least squares fitted with this function (FIG. 9, solid lines).

The bulk flow velocities applied in this study resulted in relatively high speeds of flow at the level of settled cells. Cell travel velocities at 140 μm/s, or even at 60 μm/s, is generally significantly faster than velocities which are optimal for cell navigation where the pistons are actuated manually (typically, from 5 to 50 μm/s) when precise cell patterns are to be formed on the micrometer scale. Yet, it is obvious from the results (FIG. 9) that cells can attach onto the studied surfaces after only a few minutes with strengths sufficient to withstand these strong local flows, albeit showing highly distinguishable attachment strengths between the three studied substrates. This is despite the statistical nature of the process of cell attachment. It is obvious from the results that Si/SiO₂ allows for the strongest attachment and cell-to-polyimide attachment is the weakest. Higher flow speed requires longer contact time to achieve the same strength of attachment.

Due to normalization, the time constants, can be used alone to characterize attachment kinetics. Smaller τ values mean stronger and faster attachment. For example, the time constants for cells on Si/SiO₂ were about five times less than those on polyimide, meaning that the former is much more suitable for cell attachment sites.

Regression coefficients of each fit, r², span a range from 0.83 to 0.93, meaning that each fit is reasonably good in a statistical sense. In addition, standard error of the time constant, Δτ, are very low relative to the differences in τ between the trends that are to be compared (FIG. 2). For example, in the results that might look like the most “scattered”, i.e., in the polyimide data, τ=1.83±0.39 for the higher flow rate, while τ=5.05±0.51 for the slower flow. Although the standard errors are indeed quite significant with respect to the respective estimated values (e.g., 0.39 compared to 1.83 for the faster flow), they are small compared to the differences (between 1.83 and 5.05). The situation is similar when we compare different substrates at the same flow rate; e.g., 1.11, 2.56, and 5.05 are the estimated time constants for the faster flow. These values are comfortably apart from each other relative to the respective standard errors to render the different trends distinct.

It should also be noted that each data point (i.e., the probability of cells staying attached) in the graphs was obtained from more than a hundred cell samples, i.e., more than a hundred independent observations. The relatively large uncertainties in the observed trends seem to be because different cells have different sizes and shapes, and their adsorption depends on these variables as well as the actual part of the cell that gets attached.

### Example 5

### Manipulation and Velocity Tests

Platforms are prepared using techniques described for Example 1 with a polyimide layer 48 on a surface-oxidized silicon (Si/SiO₂) substrate with an Si/SiO₂ opening 60 of about 20 μm.

Using the apparatus of FIG. 1, manipulation experiments are carried out. 20 μL of PBS solution with suspended cells (approximately 10⁵ cells/mL) are added to the PBS media 80 covering the platform, close to the Si/SiO₂ opening (trapping site) by a micropipette (Gilson, 5-20 μL variable). After waiting for all cells to settle down on the substrate (10-20 s), which is observed by tuning the microscope focus to just above the substrate, the experiment is initiated.

A selected individual target cell is navigated to the Si/SiO₂ opening by controlling (pushing or pulling with an appropriate speed) the x- and y-syringes. Five minutes later, also by controlling the syringes to provide an optimum cell cleaning rate (V_{cell}≈5-50 m/s), all other unwanted cells settled on the surrounding polyimide surface are moved away while keeping the target cell (or cells) on the Si/SiO₂ opening attached.

### Example 6

### Cell Movement Correlation to Syringe Operation

Using the procedure described for Example 5, correlation between average flow velocity at the exit of the tube and induced cell velocity is explored by pushing a syringe at several different constant rates. At each rate, the distance that a settled cell traveled on polyimide during the time of observation is measured under the microscope, and cell travel speed (V_{cell}) is calculated. The volume pushed out from the tube during this interval is also read from the syringe measure, to calculate the volumetric flow rate (Q) and the average flow velocity (Vₐᵥ) at the outlet of the tube.

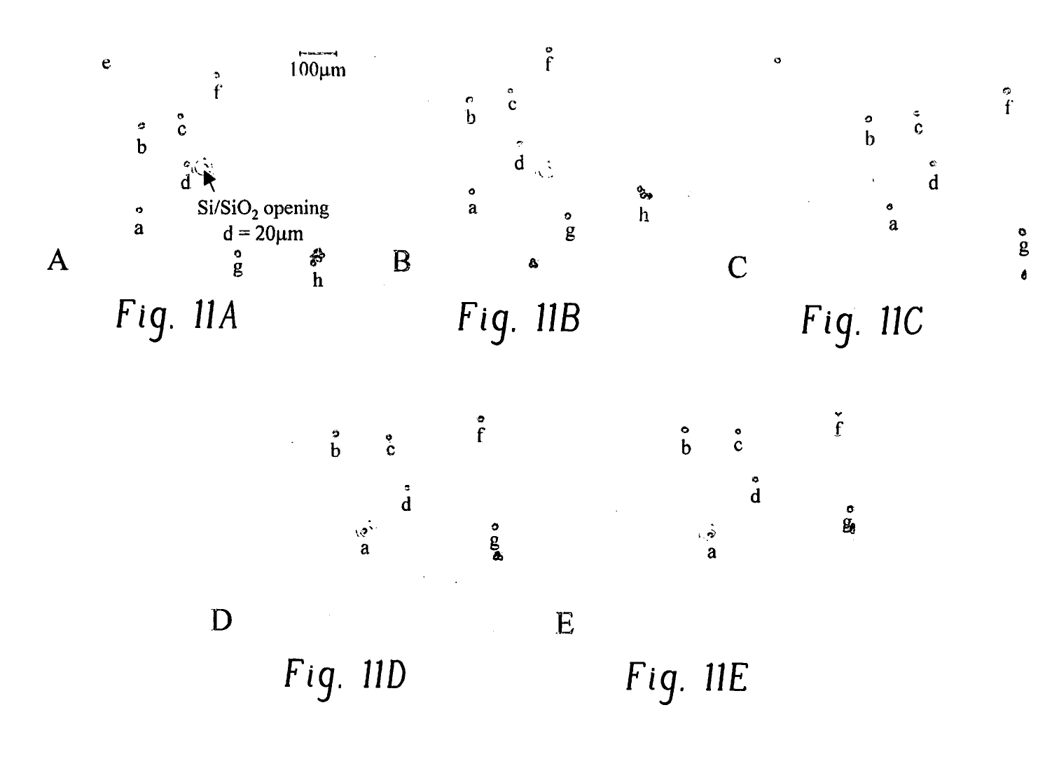

Movements of cells settled on polyimide surface at several syringe pushing speeds were recorded. FIG. 10 shows the average manipulated cell velocity vs. the average liquid flow velocity at the outlet of the tube. An experimental correlation (solid line) between the average flow velocity at the outlet of the tube and the manipulated cell velocity on polyimide observed under the microscope (□) is shown. Dashed lines represent the boundaries of the 95% confidence interval around the linear regression line. Regression between the average flow velocity at the outlet of the tube and the observed cell velocities resulted in reasonable linearity (FIG. 10, r²=0.74).

This flow calibration was compared with cell velocity derived from visually observed flow patterns in FIG. 8D. The syringe is operated, in typical cell manipulation, at speeds similar to those corresponding to FIG. 8B-D (0.1-3.0 cm/s, slower velocities being used for fine adjustments). However, only the images in FIG. 8D could be used to estimate V(−5,0) and V(0,0), due to limitation in the available temporal resolution at the faster flow rates. The flow patterns yield V(−5,0)≈0.19 cm/s and V(0,0)≈0.12 cm/s, resulting in Vₐᵥ=(½) V(−5,0)≈0.10 cm/s (Q=1.5 μL/s) and V_{cell}≈(½) V(0,−0.99) V(0,0)(1-|−0.99|^{1.5})=9 μm/s, respectively. (Here, z=−0.99 mm is used because the approximate cell size is 10 μm.) This result, indicated in FIG. 10 as □, lies well within the 95% confidence interval of the straight line fitted to experimental results from actual cell navigation experiments. This lends support to the simple semi-quantitative theoretical considerations outlined above.

It can be seen that there is a generally linear relationship between flow velocity and cell velocity with the average flow velocity of the liquid at the tube outlet Vₐᵥ being approximately 150 times that of the manipulated cell velocity V_{cell}.

### Example 7

### Selective Movement and Patterned Attachment of Cells

A manipulation system as described in Example 2 is used to study the movement of a group of individual cells as described for Example 5.

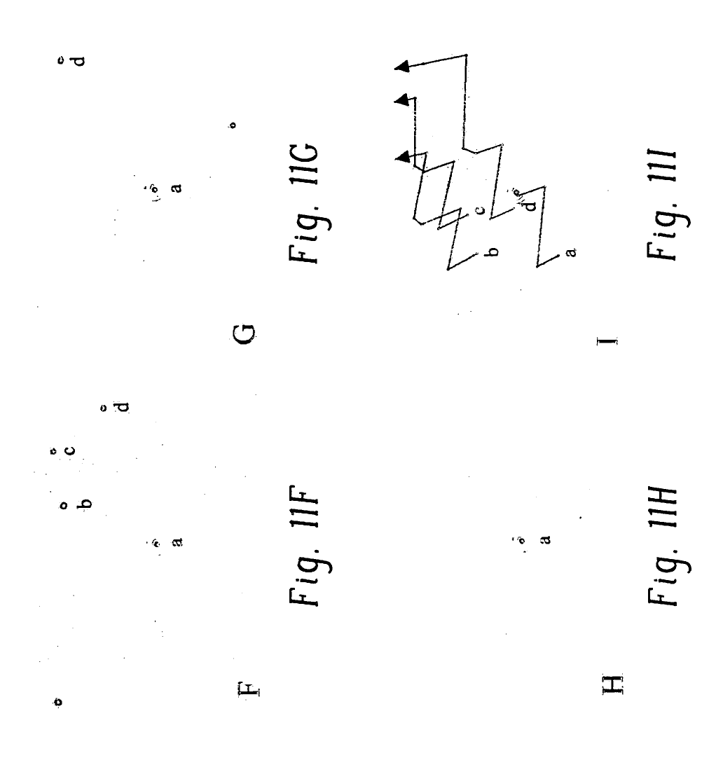

FIG. 11 shows sequential pictures of navigated and then trapped cells, obtained with the hydrodynamic micromanipulation system. Individual cells are identified and tracked by corresponding lower case letters (a-h). Cells a-h are micromanipulated by the x- and y-syringes (positioned below and to the left of this figure, respectively), aiming cell a to be attached onto the Si/SiO₂ opening site while rejecting all other cells. The same lower case letters (a-h) label the same cells in each picture. FIG. 11A shows starting conditions after the cells settled on the chip. Each subsequent picture (FIGS. 11B-E) shows conditions after one syringe operation step for navigation. After waiting for five minutes, all cells except cell a are moved away (FIG. 11F-H). FIG. 11I shows the resulting trajectories of the individual cells (a-d) during the entire cell manipulation process.

In the sequence, the chosen target cell (a) was navigated in consecutive discrete steps onto a Si/SiO₂ opening of 20 μm diameter by sequentially pushing the x- and y-syringes. The other cells (b-h) were, of course, also moved parallel to the trajectory of cell a. It required about one minute at the most to perform all these navigating steps (FIGS. 11A-E). After about five minutes of waiting, those cells that settled on the polyimide surface were removed in the cleaning steps (FIG. 11 FIG. 11), while the initially chosen target cell stayed trapped on the desired cell site (FIG. 11H). It took less than seven minutes to accomplish all these procedures. About five minutes of waiting between the navigating steps and the cleaning step(s) was sufficient for keeping the defined target cell(s) attached on the opening while removing all other cells from the surrounding polyimide surface.

FIG. 11I depicts the entire trajectory of cells a-d from which it is obvious that the x- and y-axes of cell travel lines were not exactly perpendicular (inclined at about 56° angle). Although this did not prevent the selected cell from reaching the target site, a more orthogonal arrangement of the two tube openings is preferred since this can ensure optimum efficacy, i.e., to achieve the end result in the smallest number of steps.

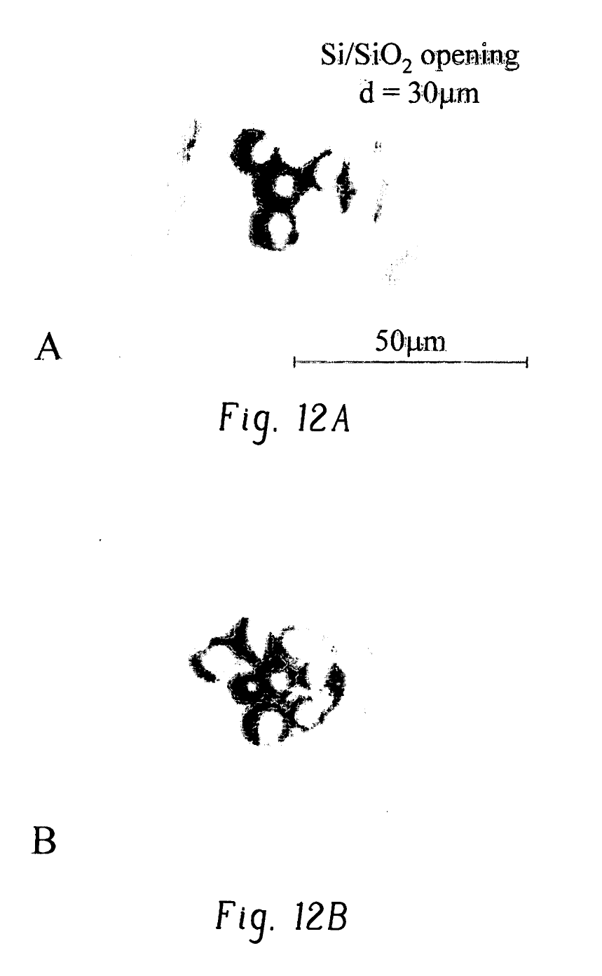

Repeating this single cell navigating, trapping, and cleaning procedure, several pre-selected desired formations of small, clearly defined cell clusters were obtained. In these experiments, a 30 μm diameter cell attachment site was used to form the desired monolayer patterns. Two examples are shown in FIG. 12, a Y shape (FIG. 12A); and densely packed cells (FIG. 12B). It took about 30 and 50 minutes, respectively, for the entire procedure to achieve the desired patterns.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

1. A method of manipulating a cell comprising:
a) delivering at least one cell to a liquid medium which extends over a surface;
b) applying a first fluid flow to the liquid medium from a first direction which moves the cell in a first direction on the surface; and
c) applying a second fluid flow to the liquid medium from a second direction angularly spaced from the first direction which moves the cell in a second direction on the surface.

2. The method of claim 1, further including, prior to steps b) and c), allowing the at least one cell to settle on the surface.

3. The method of claim 1, further including:
repeating steps (b) and (c) as necessary to position the cell on a preselected site on the surface.

4. The method of claim 3, wherein the site is more hydrophilic than an adjacent area of the surface over which the cell is manipulated to the site.

5. The method of claim 3, further including, after the step of positioning the cell on the site:
d) cleaning other cells from the adjacent area of the surface.

6. The method of claim 5, wherein step d) includes:
applying at least one of the first and second fluid flows to move the other cells away from the site.

7. The method of claim 5, further including, prior to step d):
allowing an attachment time to elapse which is sufficient for the cell to attach to the site.

8. The method of claim 7, wherein the attachment time is up to about 5 minutes.

9. The method of claim 1 wherein step (b) includes delivering a fluid from a first fluid outlet and step c) includes delivering a fluid from a second fluid outlet into the liquid medium.

10. The method of claim 9, wherein the fluid includes a liquid.

11. The method of claim 9, wherein the step of delivering includes operating a first delivery device fluidly connected to a first tube which defines the first fluid outlet and operating a second delivery device fluidly connected to a second tube which defines the second fluid outlet.

12. The method of claim 11, wherein the step of operating includes depressing a syringe.

13. The method of claim 11, wherein an outlet end of each of the tubes is arranged generally parallel with the surface.

14. The method of claim 3, wherein the surface is defined by a platform, the platform including at least one of:
an electrode positioned adjacent the site for performing an electrochemical measurement on the cell; and
an optical sensor positioned adjacent the site which exhibits a change in an optical property in response to a change in a local concentration of a chemical species.

15. The method of claim 1, wherein at least one of the first and second fluid flows has an average velocity which is at least ten times a velocity at which the cell moves in at least one of steps b) and c).

16. The method of claim 15, wherein the first and second fluid flows each have an average velocity which is at least about 100 times the velocity at which the cell moves in steps b) and c).

17. A system for manipulation of a cell comprising:
a platform which defines a surface having a site at which the cell has a higher probability of attachment in a period of time than an adjacent area of the surface;
first means for selectively delivering a fluid to a first fluid outlet located adjacent the surface, the first outlet being oriented to provide a first fluid flow in a first direction in a liquid medium on the surface;
second means for selectively delivering a fluid to second fluid outlet located adjacent the surface, the second outlet being oriented to provide a second fluid flow in a second direction, angularly spaced from the first direction, in the liquid medium on the surface, whereby by actuation of the first and second delivery devices, a cell can be navigated across the adjacent area through the liquid medium to the site.

18. The system of claim 17, wherein the site is less than 100 micrometers in diameter.

19. The system of claim 18, wherein the site is less than about 30 micrometers in diameter.

20. The system of claim 17, wherein the site is defined by a layer of at least one of silicon and an oxide thereof.

21. The system of claim 17, wherein the site is generally at an intercept of the first and second fluid flows.

22. The system of claim 17, wherein the first means for delivering a fluid includes a first syringe fluidly connected with a first tube which defines the first outlet and the second means for delivering a fluid includes a second syringe fluidly connected with a second tube which defines the second outlet.

23. The system of claim 17, wherein the first and second fluid flows are generally orthogonal.

24. The system of claim 17, wherein the site is surrounded by the adjacent area.

26. The system of claim 17, wherein the site is spaced from the outlets by a distance of from about 1-10 mm.

27. The system of claim 17, further including: means for observing movement of cells to the site.

28. The system of claim 27, wherein the observing means includes at least one of a microscope and a CCD video camera.

29. The system of claim 17, wherein the platform further includes:
at least one electrode located adjacent the site, for performing an electrochemical measurement which is influenced by a cell attached to the site.

30. The system of claim 17, further including optical monitoring equipment for detecting changes an optically detectable property of a cell attached to the site.

31. The system of claim 17, further including:
a container which receives the platform;
a liquid medium disposed in the container and covering the platform and the first and second outlets.

32. A system for performing an electrochemical measurement on a cell, the system including:
a platform which defines a surface and at least one electrode positioned for performing an electrochemical measurement which is influenced by a cell located on the surface adjacent the electrode;
a first tube positioned to supply a liquid to a liquid medium on the surface toward the electrode in a first fluid flow direction;
a second tube positioned to supply a liquid to the liquid medium on the surface toward the electrode in a second fluid flow direction; and
means for selectively controlling fluid flow to the first and second tubes.

33. A method for forming a cell manipulation system comprising:
connecting a first syringe to a first tube;
connecting a second syringe to a second tube;
positioning an outlet of a first tube in a liquid medium adjacent an upper surface of a platform in a first orientation, the platform defining region of interest comprising a site for attachment of a cell and an area surrounding the site at which a probability of a cell attaching is less than that at the site;
positioning an outlet of a second tube in the liquid medium adjacent the upper surface of the platform in a second orientation, such that fluid flows from the first and second tubes intercept at the region of interest, whereby by selectively actuating the first and second syringes, a cell located in the liquid medium in the region of interest is navigated to the site.
